(19) United States
(12) Patent Application Publication
(10) Pub. No.: US 2013/0231692 A1
(43) Pub. Date: Sep. 5, 2013

(54) Gastric Sponge System and Use Thereof

(71) Applicants: Anthony N. Kalloo, Baltimore, MD (US); Mouen Khashab, Towson, MD (US)

(72) Inventors: Anthony N. Kalloo, Baltimore, MD (US); Mouen Khashab, Towson, MD (US)

(73) Assignee: THE JOHNS HOPKINS UNIVERSITY

(21) Appl. No.: 13/882,963

(22) PCT Filed: Nov. 1, 2011
(86) PCT No.: PCT/US11/58830

§ 371 Date: May 15, 2013

Related U.S. Application Data
(60) Provisional application No. 61/443,187, filed on Feb. 15, 2011.
(60) Provisional application No. 61/409,270, filed on Nov. 2, 2010.

(51) Int. Cl. A61F 5/00

(52) U.S. Cl. CPC A61F 5/0013
USPC 606/198

(57) ABSTRACT

The present invention provides a low-risk, unobtrusive and noninvasive method and system for treatment of obesity and eating disorders. The system includes a gastric sponge device suitable for placement in a stomach of a subject. The sponge device absorbs fluid upon implantation and expands in volume, thereby functioning as a space occupying device in the stomach to cause early satiety.

### BACKGROUND OF THE INVENTION

1. Field of the Invention

The invention relates generally to treatment of obesity and eating disorders and more specifically to a gastric sponge system for treatment of such disorders.

2. Background Information

Obesity is a complex metabolic disease that carries a severe toll of comorbid illness. Over the past few decades, obesity has evolved into a global epidemic, and it is now more prevalent than malnutrition from hunger. Obese patients are deemed to be surgery-eligible only if they were obesity class I (body mass index [BMI] >40 kg/m-) or obesity class II (BMI 35-39.9 kg/m²) with significant associated comorbid illness. Although it is clear that increasing BMI is associated with a higher burden of comorbid illness, stringent application of these well-rounded BMI categories in determining which patients are eligible for treatment leaves many without effective therapeutic options. For patients who do not meet these criteria, lifestyle modification and medications have been recommended; however, these have been of limited effectiveness and durability, with high rates of attrition. Alternatively, surgical intervention is effective and provides durable results for many patients but with a substantially higher risk profile. The reduced risk profile and unique characteristics of endoscopic obesity procedures may allow the introduction of new categories of procedures with different points of intervention. Thus, a need exists for a low risk and effective strategy to assist obese patients with weight reduction.

### SUMMARY OF THE INVENTION

The present invention provides a low-risk, unobtrusive and minimally invasive method and system for treatment of obesity and eating disorders. Accordingly, in one aspect, the present invention provides a gastric sponge system. The system includes a gastric sponge device suitable for placement in a stomach of a subject to provide the subject with a sensation or perception of satiety. The sponge includes a core region; and an outer region with one or more protuberances from the core region which are compressed during delivery into the stomach and deploy on delivery to prevent migration of the device out of the stomach by passage through the pyloric valve. Additionally, the core region and outer region are composed of a biocompatible, substantially non-degradable sponge material that increases in volume upon absorption of fluid.

In another aspect, the present invention provides a method of treating obesity. The method includes introducing a gastric sponge device of the present invention into the stomach of a subject, thereby treating obesity. In one embodiment, the sponge is introduced in a compressed configuration into the stomach, preferably by endoscopic delivery. Upon contact with fluid, the sponge expands in volume to occupy up to approximately 80% of the volume of the stomach to assist in creating a feeling of satiety in the subject. In one embodiment, the sponge is pre-sized to occupy a predetermined volume of the subject's stomach by measuring the stomach volume of the subject prior to introduction of the sponge using a sizing balloon catheter. In some embodiments, the sponge is introduced in a compressed configuration into the stomach via an endoscope and may be removed from the stomach by attachment to a retrieval device inserted through the endoscope and compression of the sponge as it is retracted by the retrieval device into the endoscope.

### BRIEF DESCRIPTION OF THE DRAWINGS

FIG. 1 is a top plan view of the device of the invention with the protuberances in a deployed configuration around a central cylindrical structure.

FIG. 2 is a side view of a device of the invention with the protuberances in a partially (FIG. 2A) and fully (FIG. 2B) compressed configuration.

FIG. 3 is a cross-sectional view of a device of the invention showing two of a plurality of nubs extending from the porous outer region of the device overlaying the core region.

### DETAILED DESCRIPTION OF THE INVENTION

The present invention provides a minimally invasive and effective method for treatment of obesity and eating disorders via a gastric sponge system. The method and system have many advantages over current methodologies, such as minimal invasiveness, low cost, availability of biocompatible sponge materials, repeatability of the implantation and removal procedure, and use as a bridge to surgery (for morbidly obese patients who are not fit for surgery due to their extreme overweight). The procedure to implant the device in the stomach can be entirely performed in a minimally invasive procedure by endoscopic introduction into the stomach. Additionally, the device can be easily removed from the stomach when needed.

Before the present device and method are described, it is to be understood that this invention is not limited to the particular configuration and method described. It is also to be understood that the terminology used herein is for purposes of describing particular embodiments only, and is not intended to be limiting, since the scope of the present invention will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the invention, the preferred methods and materials are now described.

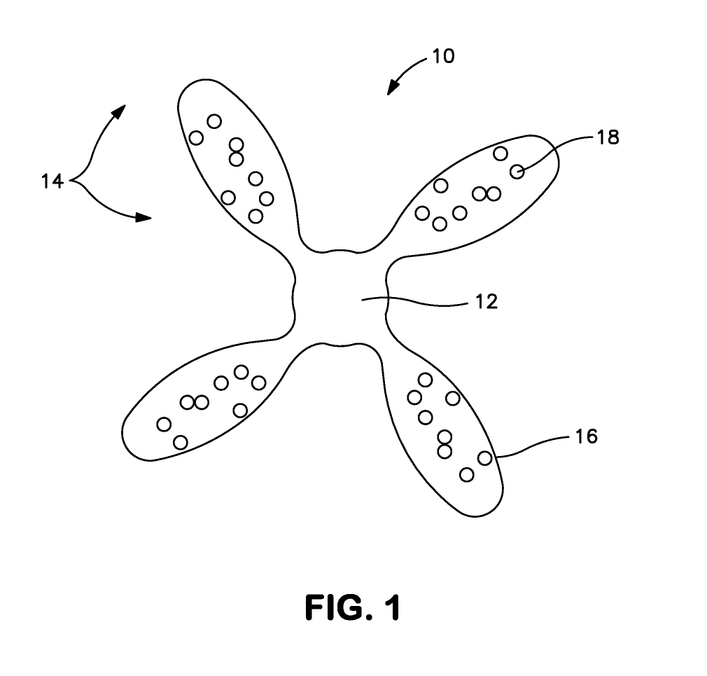

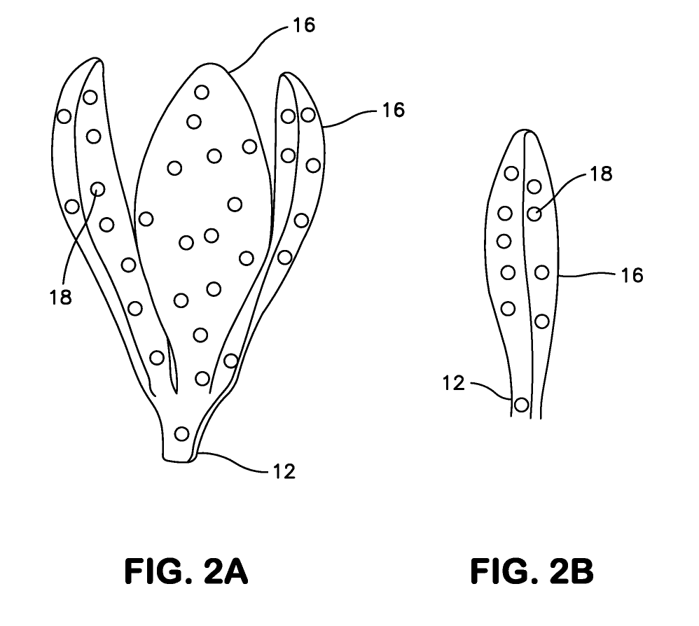

In one aspect, the present invention provides a gastric sponge system. The system includes a gastric sponge device suitable for placement in a stomach of a subject. As depicted in FIG. 1, the device 10 includes a core region 12 and an outer region 14 with one or more protuberances 16 which extend outwardly from the core region 12 when deployed in the stomach; e.g., from the proximal end of an endoscope. Both core region 12 and the surfaces of outer region 14 are porous (see, pores 18). Prior to delivery, protuberances 16 are compressed as shown in FIG. 2. For example, as shown in FIG. 2B, the protuberances can be compressed against core region 12 for insertion into or alongside an endoscope using a delivery cartridge deployable through the central lumen of a catheter and from the tip of the scope when placed at a target site, such as through the pyloric valve inₜo the stomach. Deployment of the protuberances 16 into the gastric space (from the partially compressed configuration of FIG. 2A to the fully deployed configuration depicted in FIG. 1) assists in retention of the device in the stomach by preventing its passage through the pyloric valve.

The method and device of the present invention are useful for weight loss and treatment of obesity. Generally, the gastric sponge may be configured as a through-the-scope tool or in the form of a delivery cartridge that is delivered alongside the scope, and the methods for implantation and extraction of the device are performed to completion endoscopically. The sponge device absorbs fluid upon implantation and expands in volume, thereby functioning as a space occupying device in the stomach to cause early satiety.

As such, in another aspect, the present invention provides a method of causing weight loss to treat obesity. The method includes introducing a gastric sponge device of the present invention into the stomach of a subject to cause satiety by occupying a volume of the stomach. In one embodiment, the sponge is introduced in a compressed configuration into the stomach via being loaded into a catheter and introduced through the accessory channel of an endoscope or alongside the scope and advanced into the stomach. Subsequently, the sponge expands upon hydration.

As used herein, the term “gastric sponge”, is intended to refer to a device that is formed to a size suitable for deployment in the stomach of a subject and, following deployment, expands to a size greater than the administration size upon contact with fluid. In this respect, “deployable” means that protuberances, or wing-like structures, which are axially compressed in line for delivery into the stomach unfold (e.g., in the manner of flower petals) to extend outwardly from the central core region of the device.

The term “expandable”, as used herein, means the ability of the device to expand upon hydration of the device material. As discussed in detail herein, the expandable gastric device of the invention exhibits the ability to absorb, as well as release fluid resulting in an increase and decrease in volume of the device respectively. Release of fluid and consequent reduction in volume of the device may result from external pressure or “squeezing” of the device during removal of the device or by pressure resulting from contraction of the stomach. Dynamic expansion and contraction of the sponge while present in the gastric space is especially advantageous, as the gastric adaption will not be able to occur over time; i.e., when the stomach expands to accommodate both a foreign object (e.g., a gastric balloon) and other solids (i.e., food).

In various embodiments, the core region and outer region are composed of a biocompatible, substantially non-degradable sponge material that increases in volume upon absorption of fluid. The sponge may be composed of a variety of biocompatible materials so long as the materials are substantially non-degradable and exhibit sponge like properties, for example the ability to expand and contract. Biocompatible, non-degradable sponge materials suitable for implantation include polyethylene glycol (PEG) based hydrogels, such as those described in U.S. patent application Publication No. 2009/0238815, which is incorporated herein by reference.

Other suitable materials include cross-linked chitosan sponges. Chitosan sponges may be prepared by freeze-drying of high and low molecular weight (e.g., 1.25% and 2.5% (w/w)) chitosan solutions, using a cross-linking agent, such as glutaraldehyde. The hardness and compressibility of the sponges is typically a function of the cross linking agent concentration and volume where an exemplary concentration is about 5%.

Additional suitable sponge materials include, but are not limited to, collagen materials, such as those used in tissue engineering applications (e.g., bone growth scaffolds), silicones, polytetrafluoroethylene (PTFE), polyether ether ketone (PEEK), open cell polyurethane foams and polypropylene. In one embodiment, the sponge device may be composed of material having a pore volume that is sized to only allow absorption of fluid.

In various embodiments, the sponge material is capable of expanding in volume upon hydration. As is known in the art, the increase in volume will be dependent upon the properties of the material used, but typically is capable of increasing in volume by a factor of at least 2, 3, 4, 5, 6, 7, 8, 9, or even 10 upon complete saturation with a fluid.

The sponge device 10 may be of any shape, since the sponge material is compressible to facilitate implantation. In exemplary embodiments (e.g., as shown in FIG. 2), the sponge may have a compressed configuration that is generally spherical, cylindrical or capsule-shaped (see, e.g., FIG. 2B) before deployment in the stomach. To prevent migration of the device distally through the pylorus and ensure that the device remains in the stomach until it is retrieved, the outer region includes one or more protuberances 16 which are deployable outwardly from the core region (to a deployed configuration as shown in FIG. 1) from a compressed configuration (as shown in FIG. 2) on delivery into the stomach.

In one embodiment, the device includes multiple protuberances 16 which may be disposed circumferentially around the core region 12. While various shapes of protuberances 16 may be envisioned to prevent migration of the device, in one configuration, the protuberances 16 are wing-like structures which assume an expanded configuration extending outwardly from the core region 12, much like opening of flower petals around a stem, on deployment in the stomach.

In various embodiments, the sponge device may also include regions that are treated to block the adsorption of fluid and/or food particles in specific regions of the device. Such non-adsorptive polymers include those that are widely used in medical devices as described in U.S. Pat. No. 5,169,720 and U.S. Pat. No. 5,039,458, which are incorporated herein by reference. Other suitable coatings include hydrophilic coatings that are employed on surgical devices that work by creating a water barrier as described in U.S. Pat. No. 6,238,799 and U.S. Pat. No. 6,866,936, which are incorporated herein by reference.

As such, in various embodiments, the core region of the device includes one or a plurality of raised surfaces treated to resist adsorption of food particles. The plurality of raised surfaces may be disposed in any configuration on the surface of the core region, for example, over the entire surface of the core region.

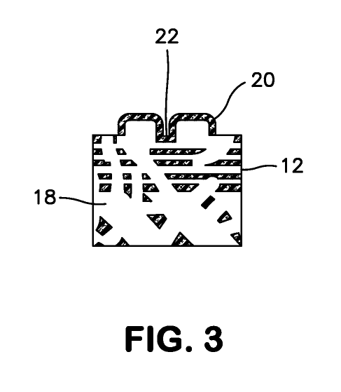

In one embodiment, the core region 12 of the device is generally cylindrical or capsule shaped. In a particularly preferred embodiment illustrated by FIG. 3, all or a majority of the outer surface of the device (including the core region 12 and protuberances 16) is covered with a plurality of raised nubs 20 treated to resist adsorption of food particles. Nubs 20 may also be treated with a coating to resist adherence of food particles thereto, and may optionally also be porous (i.e., pores 18 may be present). The portions of the outer surface 22 interposed between nubs 20 may optionally be free of surface treatment, if present.

As discussed herein, the device may be pre-sized to occupy a particular volume of the stomach upon being fully inflated. In various embodiments, the device is sized such that it occupies up to 40, 50, 60 70, 80 or 90% of the subject's stomach upon full inflation. In an exemplary embodiment, the device is sized such that it occupies about 80% of the subject's stomach upon full inflation.

To determine the appropriate size of the device for an individual subject, the size of the subject's stomach is measured before implantation of the device. A variety of techniques may be employed to determine the volume of the stomach. For example, x-ray or other types of imaging may be used. In an exemplary embodiment, the stomach volume is determined using a gastric sizing balloon catheter. The catheter includes a balloon at its distal tip. The catheter is advanced into the stomach and the gastric balloon inflated with a fluid, such as water, air, radiopaque material, or the like until it occupies the desired volume of the stomach, e.g., 80% of the total volume of the gastric lumen. The sizing procedure may be performed under endoscopic and/or radiologic guidance. The volume of fluid inserted that results in the desired occupancy of the stomach by the balloon is used to determine the size of the gastric sponge.

The gastric sponge is substantially non-degradable to facilitate retrieval of the device from the subject after a desired time. As such, the system may further include a retrieval device for attachment to the sponge. The retrieval device may be deployed through an endoscope by retracting the sponge into the endoscope causing the sponge to substantially return to its compressed configuration (e.g., as shown in FIG. 2B).

The gastric sponge may be deployed within the stomach for various durations before being retrieved. In various embodiments, the device is deployed in the stomach for about 1-7 days, 1-2 weeks, 2-4 weeks, or several months. Upon retrieval, the sponge may be disposed of and a new gastric sponge (potentially of larger or smaller size than the retrieved sponge) deployed, the process continuing until the desired weight of the subject is achieved.

Although the invention has been described with reference to the above example, it will be understood that modifications and variations are encompassed within the spirit and scope of the invention. Accordingly, the invention is limited only by the following claims.

What is claimed is:

1. A gastric sponge system comprising, a gastric sponge device suitable for placement in a stomach of a subject, the sponge comprising:
a) a core region; and
b) an outer region comprising one or more protuberances from the core region which prevent migration of the device through the pyloric valve,
wherein the core region and outer region are composed of a biocompatible, substantially non-degradable sponge material that expands in volume upon absorption of fluid.

2. The system of claim 1, wherein the material is capable of at least doubling in volume upon contact with an ingested fluid.

3. The system of claim 1, wherein the sponge has a compressed configuration that is generally spherical, cylindrical or capsule-shaped before deployment in the stomach.

4. The system of claim 3, wherein multiple said protuberances are disposed circumferentially around the core region.

5. The system of claim 4, wherein the protuberances are wing-like structures which assume an expanded configuration extending outwardly from the core region on deployment in the stomach.

6. The system of claim 1, wherein the core region further comprises a plurality of raised surfaces treated to resist adsorption of food particles.

7. The system of claim 6, wherein plurality of raised surfaces are disposed over substantially the entire surface are of the core region.

8. The system of claim 1, wherein the pore volume is sized to only allow absorption of fluids.

9. The system of claim 1, wherein the sponge is sized to occupy 80% of the volume of the stomach when fully expanded by ingested fluid.

10. The system of claim 1, wherein the sponge is adapted for placement in the stomach without substantial degradation for a duration of any period between one day and several months.

11. The system of claim 1, further comprising an endoscope for delivery of the sponge into the stomach.

12. The system of claim 1, further comprising a dilation balloon catheter.

13. The system of claim 11, further comprising a retrieval device for attachment to the sponge, wherein the retrieval device is deployed through the endoscope and for removal of the sponge from the stomach, wherein retracting the sponge into the endoscope causes the sponge to substantially return to its compressed configuration.

14. A method of treating obesity comprising introducing a gastric sponge device into the stomach of a subject, the sponge device comprising:
a) a core region; and
b) an outer region comprising one or more protuberances from the core region which are compressed during delivery into the stomach and deploy on delivery to prevent migration of the device through the pyloric valve,
wherein the core region and outer region are composed of a biocompatible, non-degradable sponge material that expands in volume upon adsorption of fluid.

15. The method of claim 14, comprising uptake of a fluid by the subject, wherein the material is capable of at least doubling in volume upon contact with an ingested fluid.

16. The method of claim 14, wherein the sponge has a compressed configuration that is generally spherical, cylindrical or capsule-shaped before deployment in the stomach.

17. The method of claim 16, wherein multiple said protuberances are disposed circumferentially around the core region.

18. The method of claim 17, wherein the protuberances are wing-like structures which assume an expanded configuration extending outwardly from the core region on deployment in the stomach.

19. The method of claim 14, wherein the core region further comprises a plurality of raised surfaces treated to resist adsorption of food particles.

20. The method of claim 14, wherein the sponge occupies up to 80% of the volume of the stomach when fully expanded by an ingested fluid.

21. The method of claim 14, further comprising removing the sponge from the stomach after a duration of any period between one day and several months.

22. The method of claim 16, wherein the sponge device is introduced in a compressed configuration into the stomach via an endoscope.

23. The method of claim 22, wherein the sponge is removed from the stomach by attachment to a retrieval device inserted through the endoscope and compression of the sponge as it is retracted by the retrieval device into the endoscope.

24. The method of claim 14, further comprising measuring the volume of the stomach prior to introduction of the sponge into the stomach.

25. The method of claim 24, wherein the volume of the stomach is determined using a sizing balloon catheter.

26. The method of claim 24, wherein the sponge is sized to occupy about 80% of the volume of the stomach when fully expanded.

27. A method of treating obesity comprising introducing a gastric sponge device into the stomach of a subject, the method comprising:
a) deploying a sizing balloon into the gastric space to measure the volume of the space;
b) selecting a gastric sponge device of a size sufficient to induce satiety in the subject; and,
c) withdrawing the sizing balloon and deploying the gastric sponge into the gastric space.

28. The method of claim 27, wherein the gastric sponge comprises:
a) a core region; and
b) an outer region comprising one or more protuberances from the core region which are compressed during delivery into the stomach and deploy on delivery to prevent migration of the device through the pyloric valve,
wherein the core region and outer region are composed of a biocompatible, non-degradable sponge material that expands in volume upon adsorption of fluid.

29. The method of claim 27, wherein the sponge occupies up to 80% of the volume of the stomach when fully expanded by an ingested fluid.
